(19) United States
(12) Patent Application Publication
(10) Pub. No.: US 2004/0172051 A1
(43) Pub. Date: Sep. 2, 2004

(54) Method and apparatus for tubal occlusion

(76) Inventor: Sundaram Ravikumar, Briarcliff Manor, NY (US)

Correspondence Address: GORDON & JACOBSON, P.C., 65 WOODS END ROAD, STAMFORD, CT, 06905, US

(21) Appl. No.: 10/692,026

(22) Filed: Oct. 23, 2003

Related U.S. Application Data
(63) Continuation-in-part of application No. PCT/US03/06195, filed on Feb. 28, 2003.

(51) Int. Cl. A61B 17/08

(52) U.S. Cl. 606/157

(57) ABSTRACT

Devices (plugs) for occluding fallopian tubes are disclosed. The plugs, or at least portions thereof, are made of elastomeric silicone or other biocompatible material and include a pilot hole which permits an insertion device to be used to insert the plug into the fallopian tube. The plugs of the present invention allow fallopian tubes to be rapidly occluded for birth control purposes with minimal adverse side effects. The plugs are also adapted to be removed from the fallopian tubes in a relatively simple procedure without adversely affecting the fallopian tubes.

This is a continuation-in-part of International Application No. PCT/US03/06195, filed on Feb. 28, 2003, which is hereby incorporated by reference herein in its entirety.

### BACKGROUND OF THE INVENTION

1. Field of the Invention

The present invention relates to medical devices that are implanted in the human body. In particular, the present invention relates to medical devices that can be used to occlude the female mammalian fallopian tubes.

2. State of the Art

It is often desired to close the fallopian tubes of women for birth control purposes. One method for closing the fallopian tubes is surgical tubal ligation, a procedure in which the uterine tubules are tied and cut or clamped through an incision made through the wall of the abdomen. When done endoscopically, the pelvic cavity must be pneumatically inflated. Aside from injury due to possible over-inflation, numerous cases of the formation of embolisms have been reported. Tubal ligation done with a laparotomy requires a surgical incision in the abdomen done under general anesthesia. Aside from permanent scar formation at the site of incision, there are reported cases of death due to anesthesia complications.

Other methods for female sterilization have been investigated. In one technique, a curable elastomeric composition (such as silicone) is transcervically injected into the fallopian tubes in an amount sufficient to fill the portion of the oviduct adjacent the uterus. The elastomeric composition is allowed to solidify to thereby nonsurgically block the tube. This technique, which is described in U.S. Pat. No. 3,805,767 to Erb, has problems associated therewith. For example, the technique is time consuming and requires a high level of technical skill both for the preparation of the silicone and for performing the procedure.

Others techniques have been proposed. For example, U.S. Pat. No. 5,601,600 to Ton discloses the placement of an occlusive wire or coil within the fallopian tubes to occlude them. The coil must be delivered into the fallopian tubes with a delivery catheter extending from the uterus into the fallopian tubes. This technique, which is being commercialized by Conceptus, Inc. under the tradename “Essure™”, is problematic because it takes a long period of time (typically on the order of 3 months) for the wire or coil to form fibrous tissue that blocks the fallopian tube.

U.S. Pat. No. 6,346,102 to Harrington et al. discloses the placement of a releasable heat generating plug within the fallopian tubes. RF energy is supplied to the heat generation plug such that the plug thermally damages the fallopian tube to cause it to constrict around the plug. After such constriction, the plug is released and left in place to occlude the fallopian tubes. The heat generating plug includes electrically conductive electrode(s), an insulator made from thermal and electrically insulating material. In an alternate embodiment, laser light is supplied to the heat generation plug such that the plug thermally damages the fallopian tube to cause it to constrict around the plug. In this embodiment, the heat generating plug includes a cylindrical heating tip made of silicone/bioglass that is loaded with dispersive particles that disperse the laser energy throughout the tip and convert the energy into heat (which thermally damages the fallopian tube to cause it to constrict around the plug). In yet another embodiment, the heat generation plug includes a piezo-electrical crystal. Electrical energy is supplied to the piezo-electrical crystal such that it emits ultrasound energy that heats up the plug and surrounding tissue (which thermally damages the fallopian tube to cause it to constrict around the plug). These devices are problematic in that the thermal damage to the fallopian tubes is difficult to control and, if not controlled properly, may impart inadvertent damage to the surrounding tissue. Moreover, because this technique damages the fallopian tube, reversal of the procedure requires complex microsurgery or may be impossible to accomplish.

Consequently, there is a need for improved devices that efficiently and effectively occlude the fallopian tubes of women for sterilization purposes with minimal adverse side effects. Preferably, the improved occlusion devices are adapted to be removed from the fallopian tubes without undue adverse effects.

### SUMMARY OF THE INVENTION

It is therefore an object of the present invention to provide improved means for occluding the fallopian tubes of a woman for birth control purposes. Occlusion of the tubes prevents male sperm from fertilizing female eggs, thus preventing conception.

It is a further object of the invention to provide fallopian tube occluding plugs and tools for inserting those plugs into a fallopian tube.

In accord with the objects of the invention, which will be discussed in more detail hereinafter, fallopian tube occlusion devices (plugs) are disclosed. The plugs, or at least portions thereof, are made of elastomeric silicone or other biocompatible material and include a pilot hole which permits an insertion device to be used to insert the plug into the fallopian tube. The plugs of the present invention allow fallopian tubes to be rapidly occluded. The plugs are also adapted to be removed from the fallopian tubes in a relatively simple procedure without adversely affecting the fallopian tubes.

Additional objects and advantages of the invention will become apparent to those skilled in the art upon reference to the detailed description taken in conjunction with the provided figures.

### BRIEF DESCRIPTION OF THE DRAWINGS

FIG. 1 is a partial view of the female reproductive system, showing the occlusion device of the present invention being inserted into a fallopian tube and prior to detachment of the occlusion device (plug).

FIG. 2 is a perspective view of an occlusion device inserted into a uterine tube portion in accordance with the present invention.

FIG. 3 is a bottom perspective view of the occlusion device of FIG. 2.

FIG. 4 is a perspective view of an insertion device in accordance with the present invention.

FIG. 5 is an exploded view of the insertion device of FIG. 4.

### DESCRIPTION OF PREFERRED EMBODIMENTS

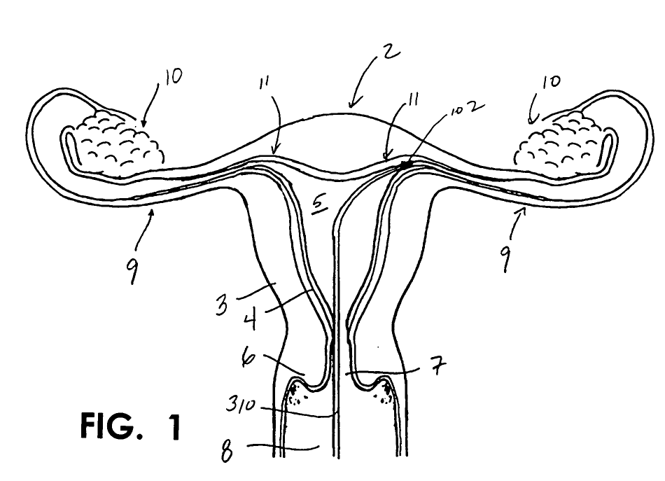

FIG. 1 shows some of the major elements of the female reproductive system. The uterus 2 is an organ of the female pelvis that has the shape of a pear. It consists of a thick muscular coat 3 (referred to as the myometrium), a cavity 4 having an inner mucosal lining of variable thickness (referred to as the endometrium), and a cavity 5 (referred to as the uterine cavity). The cervix 6 defines the cervical canal 7 which is an inferior opening to the vagina 8. The fallopian tube 9 is a bilateral duct that connects the uterus 2 to the ovary 10. The ovary 10 is the organ that produces one or more eggs during every cycle of a woman's reproductive life. Each fallopian tube 9 can be logically divided into 4 parts:

i) Infundibulum—the funnel-shaped, expanded lateral end of the tube which overlies the ovary; the free edge has many finger-like processes, the fimbriae, suspended over the ovary; it contains the abdominal ostium of the tube;

ii) Ampulla—the intermediate segment which is adjacent the infundibulum; it is relatively wide and tortuous;

iii) Isthmus—the narrow portion that lies just lateral to the uterus; and

iv) Intramural (interstitial), the uterine portion of the tube; this pierces the uterine wall, and through the uterine ostium.

The site where the fallopian tube 9 and uterus 2 connect is called the uterotubal junction 11. It is a section of tubular shape of about 10 mm in length. Its inner diameter in the resting position is less than 1 mm. But when gas or liquid is pushed through the uterus and tubes, the diameter of the uterotubal junction may stretch up to about 2 mm. The uterotubal junction 11 provides a transition between the uterus 2 and the fallopian tube 9, and the area of transition from the chamber of the uterus to the lumen of the uterotubal junction is referred to as the ostium or cornu. The ostium, uterotubal junction, and the fallopian tube itself are part of a pathway leading from the ovaries to the uterus, and this pathway is sometimes referred to as the uterine tube.

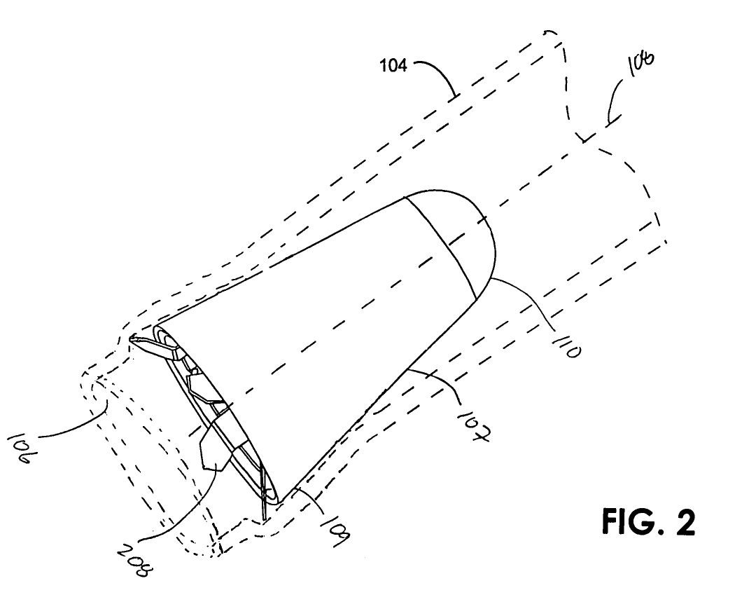

FIG. 2 illustrates a uterine tube portion 104 that has been occluded using a plug 102 in accordance with the present invention. Uterine tube portion 104 has a lumen 106 (which is part of the pathway leading from an ovary 10 to the uterus 2), and plug 102 has been inserted axially into lumen 106 to occlude the uterine tube portion. Preferably, the external surface 107 of plug 102 is symmetric about an axis 108 as shown. Furthermore, the external surface 107 preferably tapers from a base 109 disposed at one end to a rounded top portion 110 as shown. To facilitate insertion of the plug 102 into uterine tube portion 104, the cross-sectional diameter of the rounded end 110 is smaller than the diameter of lumen 106. Moreover, to ensure that the plug 102 remains at its desired position in the uterine tube portion 104, the cross-sectional diameter of the base 109 is larger (typically on the order of 10 to 35 percent larger and preferably between 25 to 30 percent larger) than the diameter of lumen 106. The plug 102 also preferably includes spring-biased prongs 208 that extend below the base 109 and radially outward with respect to axis 108 as shown.

Uterine tube portion 104 has an elastic wall surrounding lumen 106. The elastic nature of the uterine tube portion 104 allows the plug 102 to be tightly grasped by the uterine tube portion 104. More specifically, when the plug 102 is inserted into its desired position in the uterine tube portion 104, the larger diameter base 109 and possibly the spring-biased prongs 208 (which extend radially into the elastic wall of the uterine tube portion 104) cause the elastic wall of the uterine tube portion 104 to be deformed such that the plug 102 is tightly grasped by the wall of the uterine tube portion 104 as shown. Therefore, the elastic nature of the walls enables the plug 102 to be effective in occluding uterine tube portion 104. Furthermore, the elastic walls of uterine tube portion 104 also permit a small range of plugs 102 of varying diameter to be used for different sizes of uterine tube sections 104. Thus, plug 102 of a certain size may be used for occluding uterine tube sections of different sizes. Typically, plug 102 ranges from 1 mm to 2.5 mm in maximum cross-sectional diameter. When inserted into the lumen 106 of the uterine tube portion 104, the plug 102 provides total occlusion of the uterine tube portion, thereby preventing male sperm from fertilizing female eggs and thus preventing conception. Preferably, the plug 102 is inserted and positioned at or near the uterotubal junction to provide for total occlusion of the fallopian tube as shown in FIG. 1; however, it is contemplated that the plug 102 can be positioned at many locations inside the uterine tube to provide total occlusion.

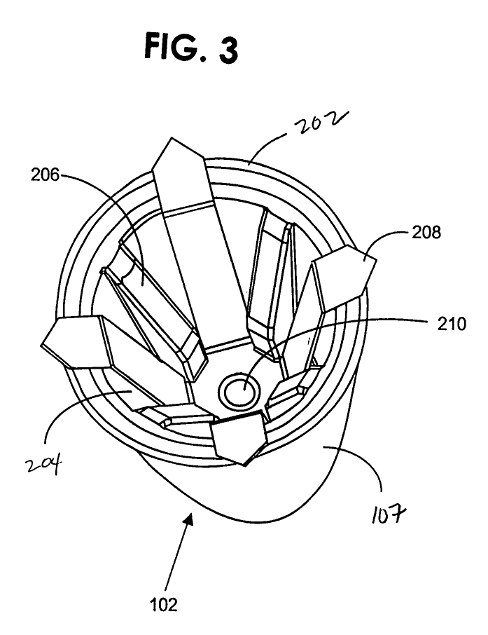

As shown in FIG. 3, the exterior surface 107 of plug 102 is formed by a layer 202 of biocompatible material. Preferably, silicone is used to form layer 202. Silicone is useful since it is non-toxic, chemically inert, substantially insoluble in blood and substantially non-immunogenic. In addition to silicone, newer elastomeric biocompatible materials may also be used to manufacture the layer 202. Ongoing research and development in biocompatible materials have created materials with a longer life, better strength and lower cost—all of which are desirable qualities of the material of the exterior surface layer 202. Typical examples of such materials include polyurethanes and polyisobutylene-based polymers.

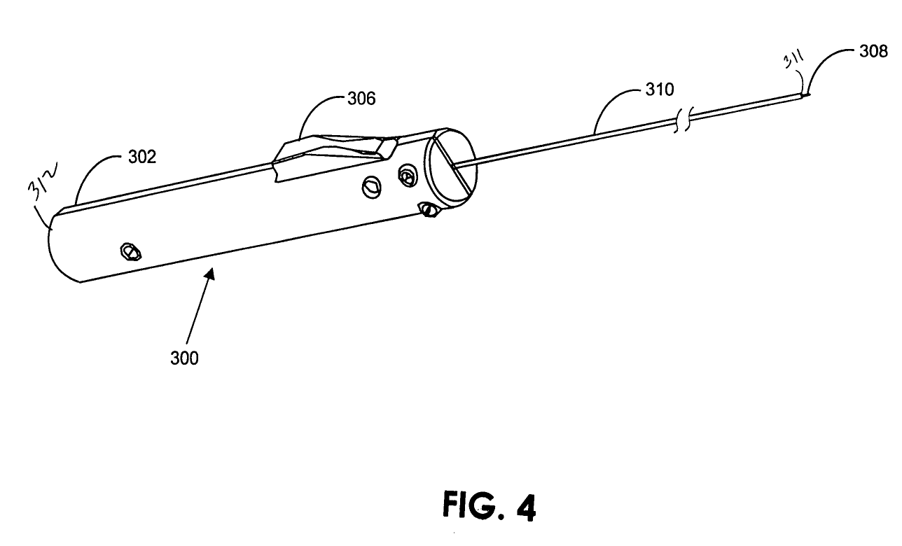

Plug 102 is preferably provided with a frame structure 204 that supports the exterior surface layer 202 such that surface layer 202 withstands the forces acting upon it when the plug 102 is inserted into the uterine tube portion 104. More specifically, upon inserting the plug 102 into uterine tube portion 104, the elastic walls of the uterine tube portion exert a compressive force on the exterior surface layer 202. The frame structure 204 mechanically supports the exterior surface layer 107 to counteract these forces. Preferably, the frame structure is corrugated and includes projections 206 that extend radially inward toward the central axis 108 as shown. These features provide for increased strength of the frame structure 204. In the exemplary plug shown, the frame structure 204 includes four projections that are spaced 90 degrees apart. The radial width of each projection tapers from a point near the top of the projection to the bottom of the projection as shown. The top of the frame structure 204 mechanically supports a prong structure which includes a plurality of spring-biased (preferably metal) prongs 208 and an integral base 209 which is joined to the prongs by joints 211. In the exemplary plug shown, the four spring-biased prongs 208 are spaced 90 degrees apart. In addition, the base 209 of the prong structure and the top of the frame structure 204 includes pilot holes 210a, 210b. These pilot holes are sized to receive an insertion device 300 and thereby enable plug 102 to be mounted on an insertion device 300 as described below with respect to FIG. 4. Preferably, the frame structure 204, including projections 206 and prongs 208, are made of hard plastic and/or metal (such as titanium) or some other material of sufficient rigidity and strength.

In an alternative embodiment (not shown) of plug 102 of FIGS. 2 and 3, the plug 102 may be constructed without any prongs 208. In this embodiment, plug 102 may be manufactured using materials of sufficient structural rigidity and strength. Furthermore, the prongs 208 may be avoided by suitably increasing the thickness of the frame structure 204.

In a typical procedure, the plug 102 is inserted into uterine tube portion 104 by substantially aligning the axis 108 of the plug 102 with the longitudinal axis of the uterine tube portion 104, and by applying a force along the axis 108. The practitioner may apply this force by using a deployment mechanism such as the insertion device 300 shown in FIG. 4. This insertion device 300 has a casing 302 and a lever 306. The practitioner uses the lever 306 to operate the insertion device 300. Lever 306 enables a needle 308, housed in a tubular needle guard 310, to be retracted inwards. To insert the plug, the practitioner mounts a plug 102 on the needle 308 of the insertion device (if the plug is not already pre-mounted). This is done by inserting the needle 308 into the pilot holes 210 of the plug 102 such that the distal end 311 of the needle guard butts up against the base 209 of the prong structure. Next, the practitioner inserts the plug/needle guard 310 into the body non-invasively: through the vagina, through the cervix and into the uterine cavity. The practitioner aligns the axis 108 of the plug 102 with the longitudinal axis of the uterine tube portion 104, and applies an axial force to the insertion device 300, thereby inserting the needle 308 and the plug 102 mounted thereon to a desired position in uterine tube 104. As the plug is pushed in the lumen of the uterine tube, the downward angle of the ends of the prongs 208 enables the prongs 208 and plug body (107, 204, 206) to slide along the walls of the uterine tube. In addition, the constricting force of the uterine tube compresses the plug body (107, 204, 206), which in turn causes the ends of the prongs 208 to be retracted inward with respect to the central axis. In this manner, any damage caused by the prongs 208 to the inner walls of the uterine tube is limited. When force is no longer applied by the practitioner, the prongs 208 spring outwardly and dig into the walls of the uterine tube. The prongs 208 together with the relative diameters of the plug 102 and lumen of the uterine tube 104 enable the elastic walls of the uterine tube 104 to tightly grip and secure the plug 102 as described above. The practitioner then presses button 306 to retract the needle 308 inward such that it is housed in the needle guard 310 and becomes disengaged from the pilot holes 210, thereby releasing the plug 102 from the insertion device 300. This procedure can be done under guidance provided by x-ray imaging, sonar imaging, an hysteroscope or blindly.

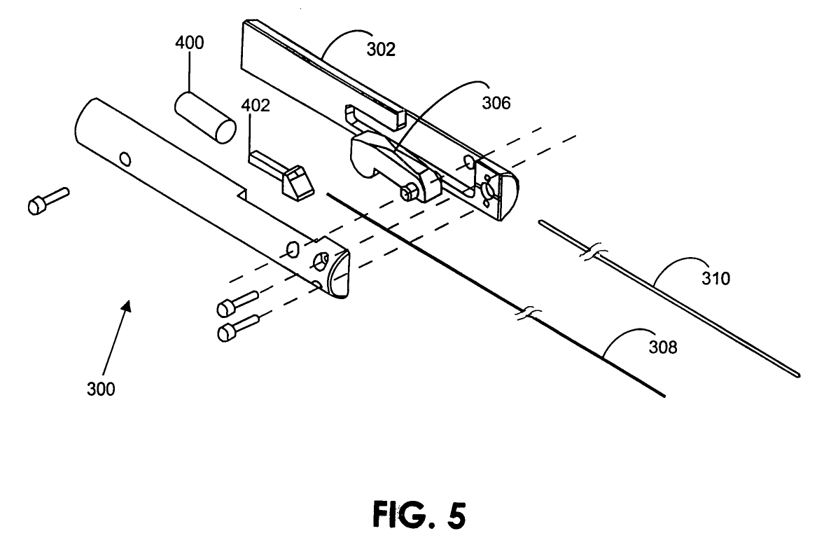

FIG. 5 shows an exploded view of insertion device 300. This device is essentially a spring activated device. A spring 400 and a needle guide 402 have been shown encased in casing 302. When the lever 306 is activated, it propels needle guide 402 toward the back end 312. In turn, this needle guide propels the needle 308 inwards such that it retracts into the distal end 311 of needle guard 310. Hence, the needle 308 is released from plug 102. When the lever 306 is not activated, the spring 400 propels needle guide 402 such that it moves away from the back end 312. In turn, the needle guide propels needle 308 outward such that it is extends from the distal end 311 of guard 310. It will be apparent to one skilled in the art that alternative ways to propel the needle may be employed by the insertion device 300. A lock may be added to deactivate the lever 306 and needle guide 402 such that once the lever 306 has been activated to deploy the plug, the needle 308 remains inside and shrouded by the guard 310 to avoid accidental trauma to the patient, practitioner or nurse. As is readily apparent from FIG. 1, the needle guard 310 (and needle 308 housed therein) is preferably malleable such that it can be readily maneuvered and bent (if necessary) prior to and/or during insertion into the uterus.

The plug device 102 of FIGS. 2 and 3 is designed to provide a permanent occlusion of an uterine tube portion 104, thereby preventing male sperm from fertilizing female eggs and thus preventing conception. In other words, it generally is not meant to be removed. Such a design is useful as a permanent birth control mechanism. However, in certain circumstances, it may be desirable to provide a mechanism to remove the “permanent” occlusion device. This may be accomplished by pushing on the base 209 of the prong structure 208 (e.g., by inserting a rigid needle/guide structure into pilot holes 210 and pushing with axial force). This will cause the spring-biased prongs 208 to retract radially inward. A relatively rigid sleeve may then be slipped over the retracted prongs so that the prongs 208 do not impale the arterial walls and so that the plug 102 may be pulled from the lumen 106 of the uterine tube portion 104.

While the present invention has been discussed above in connection with human sterilization and birth control, it will be apparent to those skilled in the art that it may also be applied in numerous animals for which sterilization/birth control is desired. While particular embodiments of the invention have been illustrated and described, it is not intended that the invention be limited thereto, as it is intended that the invention be as broad in scope as the art will allow and that the specification be read likewise. Thus, while particular materials have been disclosed, it will be appreciated that other materials can be used as well. In addition, while particular sizes of plugs have been disclosed, it will be understood that different sized plugs can be used for use in certain blood vessels. Further, while the plugs have been disclosed with reference to relative direction (e.g., top, bottom, front, rear, etc.), it will be understood that these terms are relative terms and not intended to be limiting with respect to the orientation of the plugs in space. In addition, while the plugs have been disclosed as being provided separately from an insertion device, it will be appreciated that the plugs may be pre-mounted on insertion devices, and sets of plugs and insertion devices may be sold as a kit for a particular surgery. Numerous other modifications, changes, variations, substitutions and equivalents will be apparent to those skilled in the art. It will therefore be appreciated by those skilled in the art that those modifications, changes, variations, substitutions and equivalents could be made to the provided invention without deviating from its spirit and scope as claimed.

What is claimed is:

1. A device for occluding a uterine tube portion having an interior diameter, comprising:
a body comprising a forward section having a tapered outer surface, and a flexible large diameter section adjacent said forward section having a diameter greater than the interior diameter of the uterine tube portion; and
an insert disposed along a central axis of said body and mechanically attached to said body.

2. The device according to claim 1, wherein:
said insert is formed from a hard material relative to said body.

3. The device according to claim 1, wherein:
said insert includes a plurality of barbs which engage said uterine tube portion.

4. The device according to claim 1, which is inserted into the blood vessel by use of an insertion device, wherein:
said insert has a longitudinal axis and a pilot hole along said longitudinal axis for receiving the insertion device.

5. The device according to claim 1, wherein:
said body comprises an elastomeric biocompatible material.

6. The device according to claim 5, wherein:
said elastomeric biocompatible material comprises silicon.

7. Apparatus for occluding a blood vessel having an interior diameter, comprising:
a) a plug device including
(i) a body comprising a forward section having a tapered outer surface, and a flexible large diameter section adjacent said forward section having a diameter greater than the interior diameter of the blood vessel, and
(ii) an insert formed of hard material relative to said body, said insert extending along a longitudinal axis of said plug device; and
b) an insertion device having means to attach said insert to the insertion device.

8. The apparatus according to claim 7, wherein:
said insert has a pilot hole which receives said means to attach.

9. An apparatus according to claim 8, wherein:
said insertion device comprises a needle, a tubular needle guard surrounding said needle, said needle fitting into said pilot hole of said body, and a spring-biased lever operable to retract said needle into said tubular needle guard.

10. An apparatus according to claim 7, wherein:
said plug device is mounted on said insertion device.

11. An apparatus according to claim 7, comprising:
a plurality of said plug devices.

12. A device for occluding a uterine tube portion having an interior diameter, comprising:
a body comprising a tapered outer surface and a flexible large diameter section having a diameter greater than the interior diameter of the uterine tube portion; and
an insert into said body comprising a plurality of spring-biased prongs extending beyond said body and radially outward with respect to a central axis of said device.

13. The device according to claim 12, wherein:
said body includes an exterior surface layer of elastomeric biocompatible material supported by an interior frame structure.

14. The device according to claim 13, wherein:
said frame structure includes projections that extend radially inward toward said central axis of said device, said insert being held in place by said projections.

15. The device according to claim 12, wherein:
said elastomeric biocompatible material comprises silicone.

16. The device according to claim 12 which is inserted into the uterine tube portion by use of an insertion device, wherein:
said insert includes a pilot hole for receiving said insertion device.

17. A method of occluding a uterine tube portion having an interior diameter, the method comprising:
providing a plug body comprising a tapered outer surface and a large diameter section having a diameter greater than the interior diameter of the uterine tube portion, said plug body having a central axis;
substantially aligning said central axis of said plug body with a longitudinal axis of the uterine tube portion;
applying an axial force to insert said plug body into said uterine tube portion; and
removing said axial force such that a wall section of uterine tube portion grasps onto said plug body to hold it in place.

18. A method of occluding a uterine tube portion according to claim 17, wherein:
the axial force is applied with an insertion device having means to attach said plug body to the insertion device.

19. A method of occluding a uterine tube portion according to claim 18, wherein:
the insertion device comprises a needle, a tubular needle guard surrounding the needle, the tubular needle guard fitting into a pilot hole of the plug body, and a spring-biased lever operable to retract the needle into the tubular needle guard to release an attached plug body,
the method further comprising the step of operating said lever to release said plug body.
